(19) United States
(12) Patent Application Publication
(10) Pub. No.: US 2008/0213233 A1
(43) Pub. Date: Sep. 4, 2008

(54) Method of Delivering Nucleic Acid Molecules Into Embryonic Stem Cells Using Baculoviral Vectors

(76) Inventors: Shu Wang, Singapore (SG); Jieming Zeng, Singapore (SG)

Correspondence Address: KLARQUIST SPARKMAN, LLP, 121 SW SALMON STREET, SUITE 1600, PORTLAND, OR, 97204, US

(21) Appl. No.: 11/915,627

(22) PCT Filed: May 26, 2006
(86) PCT No.: PCT/SG06/00133

§ 371 Date: Nov. 27, 2007

Related U.S. Application Data
(60) Provisional application No. 60/684,958, filed on May 27, 2005.

(51) Int. Cl. A61K 35/54; C12N 15/85; C12N 5/10; A61P 43/00; C12N 15/31

(52) U.S. Cl. 424/93.21; 435/455; 435/366; 536/23.7

(57) ABSTRACT

There is provided a method of delivering a nucleic acid molecule to an embryonic stem cell, including a human embryonic stem cell, by infecting the embryonic stem cell with a baculoviral vector comprising the nucleic acid molecule. Embryonic stem cells transduced by this method are useful for treating a disease or disorder in a subject.

### CROSS-REFERENCE TO RELATED APPLICATION

This application claims benefit and priority from U.S. provisional patent application No. 60/684,958, filed on May 27, 2005, the contents of which are incorporated herein by reference.

### FIELD OF THE INVENTION

The present invention relates generally to delivery of nucleic acid molecules into stem cells, and particularly to delivery of nucleic acid molecules by viral vectors into embryonic stem cells.

### BACKGROUND OF THE INVENTION

Versatile gene transfer vectors capable of mediating either transient or prolonged expression are required in genetic engineering of embryonic stem (ES) cells for the purposes of both regenerative medicine and basic biological studies. Furthermore, when a gene is delivered to a stem cell for the purpose of integration into the cellular genome, chromosome integration at a defined genetic locus is generally preferred compared to random integration for stable transgene expression.

Genetic manipulation of mouse embryonic stem (mES) cells has been a powerful enabling method for researchers to understand basic biological functions of many genes and genetic basis of many diseases (Downing and Battey, 2004). Unlike mouse ES cells, human embryonic stem (hES) cells have proved refractory to gene transfer with commonly used approaches.

Non-viral methods like chemical-based plasmid delivery and electroporation to introduce transgenes into hES cells have been tested for genetic manipulation of hES cells. Although providing only low levels of transfection efficiency and transient gene expression (Eiges et al. 2001), these methods, when used together with an antibiotic-resistance gene and drug selection, are able to produce stable clones of hES cells with random chromosome integration of the transgenes.

However, chemical-based gene transfection methods have generally exhibited low gene transfer efficiency in hES and tend to be inefficient for homologous recombination in hES cells (Zwaka and Thomson, 2003).

Electroporation, a method of choice to introduce foreign DNA into mES cells, can be adopted for gene transfection of hES cells (Zwaka and Thomson, 2003). With nucleofector technology, an electroporation-based method using specific transfection solution and electric parameters to deliver plasmid DNA into the cell nucleus, a transfection rate of 20% could be achieved in hES cells (Lakshmipathy et al., 2004). However, electroporation protocols that give satisfactory transfection results are usually detrimental to cells and hES cells do not survive the procedure well (Eiges et al., 2001).

Viral methods for delivery of transgenes to ES cells are also being explored. HIV-1 based lentiviral vectors were the first viral vectors employed to genetically engineer hES cells (Pfeifer et al. 2002). Lentiviral vectors have been shown to infect hES cells effectively, providing a large percentage of infected cells and stable transgene expression during prolonged undifferentiated proliferation in vitro for at least 38 weeks (Ma et al., 2003; Gropp et al., 2003). These viral vectors integrate into the host genome and are resistant to transcriptional silencing, allowing stable transgene expression (Pfeifer et al. 2002; Gropp et al., 2003; Ma et al., 2003). However, due to severe pathogenic effects of HIV-1 replication in humans, HIV-1 based vectors trigger concerns over their safety in future clinical situations, namely the emergence of replication-competent retrovirus. Also, random chromosome integration of lentivirus poses the risk of insertional mutagenesis, oncogene activation and cellular transformation. Development of leukemia in two children after gene therapy of SCID-X1 (Hacein-Bey-Abina, et al. 2003) has brought extensive attention to such a risk.

Other viral vectors such as adenoviral and adeno-associated viral vectors that have been tested in hES cells have a much lower risk of insertional mutagenesis, but their transduction efficiencies were less satisfactory (Smith-Arica et al. 2003). Although application potentials of these viral vectors are significant, concerns still remain over their safety profiles in medical treatments, especially on endogenous virus recombination, oncogenic chromosome insertion associated with random integration, non-selective cytotoxicity and pre-existing immune response against the viruses. Furthermore, gene capacities of these viral vectors, with respect to the size of insert these vectors can carry, may be a limiting factor for certain types of experiments.

Thus, there exists a need for a method for delivering transgenes to ES cells in an efficient manner, which allows for transfer of a large volume of genetic material, and which provides for either transient expression or stable integration of the transgene in the host ES cell.

### SUMMARY OF THE INVENTION

In one aspect, there is provided a method of delivering a nucleic acid molecule to an embryonic stem cell, comprising infecting the embryonic stem cell with a baculoviral vector, the baculoviral vector comprising the nucleic acid molecule.

In another aspect, there is provided a method of treating a disorder characterized by the premature death or malfunction of a specific cell type comprising administering to a subject an embryonic stem cell comprising a recombinant baculoviral nucleic acid.

In a further aspect, there is provided a recombinant baculoviral nucleic acid comprising a promoter specific to embryonic stem cells.

In still a further aspect, there is provided an embryonic stem cell comprising a recombinant baculoviral nucleic acid described herein.

There is also provided use of an embryonic stem cell comprising a recombinant baculoviral nucleic acid for treating a disorder in a subject, the disorder characterized by the premature death or malfunction of a specific cell type in the subject, including use of an embryonic stem cell comprising a recombinant baculoviral nucleic acid in the manufacture of a medicament for treating a disorder in a subject, the disorder characterized by the premature death or malfunction of a specific cell type in the subject.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

In the figures, which illustrate, by way of example only, embodiments of the present invention,

FIG. 1 is a fluorescence (top) and a phase-contrast (bottom) microscopy image of mES cells infected with recombinant baculovirus containing the eGFP gene under control of the CMV promoter at MOI of 500, on day 3 following infection;

FIG. 2 is overlappings of phase-contrast and fluorescence microscopy images (right) and fluorescence microscopy images (left) of a hES cell colony on day 1 after infection with recombinant baculovirus containing the eGFP gene under control of the CMV promoter at MOI of 200;

FIG. 3 is phase-contrast images (left) and fluorescence images (right) of a hES cell colony from day 1 to 5 after infection with recombinant baculovirus containing the eGFP gene under control of the CMV promoter at MOI of 100 and subsequent replating onto fresh mEF feeder cells;

FIG. 4 is phase-contrast images (top left), fluorescence images (top right) and overlapping images (top middle) of hES cell colonies grown from hES cell clump infected with the hybrid baculoviruses at a MOI of 10 pfu. The eGFP positive hES cells were selected and replated on fresh mEF feeder cells for 6 and 7 days (middle and bottom three images);

FIG. 5 is phase-contrast images of neurons derived from hES cells that have been infected by baculoviral vectors at an MOI of 100 pfu;

FIG. 6 is phase-contrast (A, C, E) and fluorescence images (B, D, F) showing the growth of a hES cell clump following transduction with recombinant baculovirus carrying eGFP gene under the control of the EF1α promoter, and flow cytometric analysis of the percentage of eGFP positive hES cells at 2 days post-transduction (G);

FIG. 7 is phase-contrast (left) and fluorescence images (right) (for A-C) of hES cell colonies transduced with recombinant baculoviruses carrying eGFP gene under the control of the CMV promoter at various MOIs from 1 to 100 pfu, and flow cytometric analysis of the percentage of eGFP positive hES cells at 1 day post-transduction (D);

FIG. 8 is photographs of: the RT-PCR detection of various molecular marker expression of in mock-transduced hES cells (A) and hES cells transduced by baculoviral vectors (B); the immunostaining detection of SSEA-4 and Oct-4 expression in mock-transduced hES cells (C, D) and hES cells transduced by baculoviral vectors (E, F); and the RT-PCR detection of the markers for three germ layers in embryoid bodies derived from mock-transduced hES cells (G) and from hES cells transduced by baculoviral vectors (H);

FIG. 9 is phase-contrast (left) and fluorescence (right) images demonstrating the enrichment of eGFP positive hES cells in the colony after 1st, 3rd and 7th mechanical selection. The eGFP positive hES cells were produced by transducing with hybrid baculoviruses containing the AAV rep gene and ITRs; the Rep/ITR construct that directs transgene integration is shown on the top; and

FIG. 10 is (A) a fluorescence image of the overgrowth of eGFP positive hES cells on feeders for 4 weeks without subculture; (B) a fluorescence image of neural sphere generated from the overgrown hES cells; (C) a phase-contrast image and (D) a fluorescence image of typical neural differentiation from neural sphere derived from the stable hES cells after plating for 2 weeks; and (E & F) fluorescence images of typical neurons derived from the stably transduced hES cells with eGFP in both their cell bodies and neurites.

### DETAILED DESCRIPTION

Embryonic stem (ES) cells are currently the subject of intensive research, particularly given their the ability to proliferate indefinitely and differentiate into all types of adult cells and their potential as a renewable cell source for regenerative medicine, for pharmaceutical research and development, and for basic developmental biological studies. The ability to genetically manipulate these cells using an effective nucleic acid transfer strategy is essential to realize the remarkable potential of ES cells. Possible benefits of such genetic manipulation include controlling differentiation of ES cells, isolating pure populations of specific types of ES cell-derived cells, altering antigenicity of cells to overcome immune rejection problem in transplantation medicine, and providing cell sources with new functional properties to combat specific diseases in the course of ex vivo therapy.

Embryonic stem cells, particularly human embryonic stem cells are recalcitrant to transfection with foreign DNA. However, the inventors have surprisingly discovered that embryonic stem cells, including human embryonic stem cells, can be transfected with foreign DNA by infecting the cells with a baculoviral vector that has been genetically modified to include a transgene of interest.

Thus, the present invention relates to the finding that baculoviral vectors can be used to efficiently transduce ES cells. Depending on the design of the vector, transient expression of a transgene as well as site-specific chromosomal incorporation of the transgene in ES cells can be achieved.

Thus, in the present methods, using baculoviral vectors with a promoter for an ES cell-specific gene, expression of a reporter gene or an antibiotic resistance gene in hES cells can be used to separate undifferentiated cells from spontaneously differentiated ones, thus facilitating the maintenance of pluripotent ES cells with their undifferentiated phenotype (Eiges et al., 2001). Using baculoviral vectors with a cell-specific promoter for a type of differentiated cells, genetic manipulation would allow the selection of a desired type of cells derived from ES cells and the elimination of undifferentiated cells, which are potentially tumorigenic upon cell transplantation in the body. Likewise, baculoviral vectors could be used to control differentiation of ES cells by delivering and driving the expression of a master gene that encodes a transcription factor responsible for differentiation into a specific cell lineage. When a baculoviral vector with a constitutive promoter from a housekeeping gene is used, transient or stable transgene expression in both ES cells and its differentiated progenies can be achieved.

The insect baculovirus Autographa californica multiple nucleopolyhedrovirus (AcMNPV)-based vectors are recently introduced as a new type of delivery vehicle for transgene expression in mammalian cells (Kost, et al. 2005). Baculovirus has broad tropism in both proliferating and non-proliferating, quiescent cells and, with the support of a mammalian-active promoter, is capable of efficiently transferring genes of interest to diverse mammalian cell types in vitro and in vivo. The virus can enter mammalian cells but cannot express its own genes from insect-specific promoters; thus, baculoviruses are unable to replicate and express viral proteins in vertebrate cells, thus will not provoke immune responses as a consequence of in vivo expression of virally encoded genes, recombine with pre-existing viral materials nor assist the replication of other viruses in the human body. Infection of baculoviruses in mammalian cells causes no visible cytopathic effects, even at a high MOI (Shoji et al., 1997). Another attractive advantage of using baculovirus AcMNPV as a gene delivery vector is the large cloning capacity conferred by its 130 kb viral genome, which may be favorably used to deliver a large functional gene or multiple genes from a single vector. A recent paper has demonstrated the efficient transduction of human mesenchymal stem cells with baculoviral vectors (Ho et al., 2005).

Thus, there is provided a method of delivering nucleic acid molecules of interest into embryonic stem cells in which method the embryonic stem cells are infected with baculoviral vectors containing the nucleic acid molecules.

The term embryonic stem cell is used herein in accordance with the usual meaning in the art, and refers to any undifferentiated pluripotent cell isolated from or forming part of an early-stage embryo. The term includes a single cell as well as a plurality or population of cells, including in vitro, as well as cells transduced ex vivo and transplanted in vivo, unless the context clearly indicates otherwise. The cell is a mammalian embryonic stem cell, including a human embryonic stem cell. In a particular embodiment, the cell is a human embryonic stem cell.

The term baculoviral vector refers to a baculovirus that has been genetically engineered to contain additional nucleic acid sequence(s) within the viral genome, and is thus useful as a tool for delivery of recombinant nucleic acid molecules of interest to a cell that the virus can transduce, meaning the virus can deliver its genetic material into the cell. The term recombinant baculoviral nucleic acid refers to the baculovirus genetic material without the rest of the viral packaging genes that has been genetically modified to incorporate a transgene of interest.

Baculoviruses are well known and characterized and baculoviral vectors for mammalian gene transduction, including the use of Autographa californica are known (see for example, Sarkis et al PNAS 97(26)). A skilled person can readily construct any suitable baculoviral vector for use in this invention, using known molecular biology techniques. The baculoviral vector may be a recombinant baculovirus whose genome has been modified to include a nucleic acid molecule of interest, for example that is engineered to express a transgene operably linked to a promoter as discussed below. The baculovirus may be so modified using standard techniques that will be known to a skilled person, such as PCR and molecular cloning techniques. For example, baculovirus can be readily modified using commercially available cloning and expression systems such as the BAC-TO-BAC™ Baculovirus Expression system (Gibco BRL, Life Technologies, USA).

The nucleic acid molecule may be any nucleic acid molecule that is desired to be delivered to an embryonic stem cell and which can be incorporated into the baculoviral vector, for example by recombinant techniques to insert the nucleic acid molecule into the baculoviral genetic material. The nucleic acid molecule may be a nucleic acid molecule that encodes a polypeptide or protein, that is it may include a gene, including a coding region operably linked to a promoter region. Thus, the nucleic acid molecule may be or include a transgene. The term transgene refers to a gene which is foreign to baculovirus, such that, for example, reference to expression of a transgene by a baculovirus refers to expression of a gene that is foreign to the baculoviral genome. The transgene may be a therapeutic gene, a gene encoding a selectable marker or a gene encoding a detectable reporter protein or a gene encoding a protein that provides resistance to selective environmental conditions for a cell in which the transgene is expressed, for example, that provides antibiotic resistance to a cell.

The term therapeutic gene or therapeutic transgene as used herein is intended to describe broadly any gene the expression of which effects a desired result when an embryonic stem cell expressing the therapeutic transgene, or a cell differentiated or descended from such an embryonic stem cell, is administered to a subject, including for example, treatment, prevention or amelioration of a disorder in the subject. Thus, the term therapeutic gene or therapeutic transgene is intended to describe any gene the expression of which effects a desired result when the transduced cell or its progeny cell is implanted in a subject, for example, a gene involved in cell differentiation to provide a new population of a particular type of progeny cell, or a gene involved in the treatment of diabetes to improve insulin secretion or uptake, or treatment of cardiac disorders. A therapeutic protein or peptide is a protein or peptide, that when expressed in the embryonic stem cell or in a progeny cell of the embryonic stem cell, has a therapeutic effect on the cell, or which effects a desired result within the embryonic stem cell.

Thus, in a particular embodiment the nucleic acid molecule includes a transgene encoding a detectable reporter molecule or a selectable marker that allows for detection or selection of an embryonic stem cell expressing the gene. For example, the transgene may encode a gene product that is detectable visually or by fluorescence techniques, such as enhanced green fluorescence protein (eGFP), or that is detectable using immunological techniques, for example a cell surface marker or other cell surface-expressed protein that is not normally found on the surface of the embryonic stem cell in the absence of expression of the transgene, such as particular major histocompatibility complex (MHC) molecules. Alternatively, the transgene may encode a protein, for example an enzyme that can act on a substrate to produce a molecule that is detectable visually, including by fluorescence methods or immunologically, such as a luciferase gene. In one embodiment the transgene encodes the detectable enhanced green fluorescence protein.

In another embodiment the nucleic acid molecule includes a therapeutic transgene, including any gene having clinical usefulness, such as a gene encoding a gene product or protein that is involved in disease prevention or treatment, or a gene having a cell regulatory effect that is involved in disease prevention or treatment. The gene product should substitute a defective or missing gene product, protein, or cell regulatory effect in the subject, thereby enabling prevention or treatment of a disease or condition in the subject. Therapeutic genes include growth factor genes (which include genes of fibroblast growth factor gene family, nerve growth factor gene family and insulin-like growth factor genes), and anti-apoptotic genes (including genes of bcl-2 gene family).

In a further embodiment, the nucleic acid molecule may include a transgene that is a gene involved in differentiation of embryonic stem cells into a particular desired cell type. For example, the transgene may be a gene involved in regulation of a particular differentiation pathway, or may encode a transcription factor that controls expression of certain genes involved in differentiation, or may encode a signaling protein involved in a regulatory pathway involved in or that controls or directs differentiation of the embryonic stem cell into a particular differentiated, or partially differentiated cell type.

Alternatively, the nucleic acid molecule may encode an RNA molecule, for example an antisense RNA or a small interfering RNA (siRNA) molecule. The antisense RNA or siRNA may be involved in down-regulating or inhibiting or reducing expression of a gene in the embryonic stem cell, for example a gene encoding a product implicated or involved in a disease or disorder, or a gene encoding a product involved in regulation or differentiation of the embryonic stem cell.

The transgene includes a coding region operably linked to a promoter such that the transgene is under control of the promoter and expression of the transgene is driven by the promoter. The term promoter is used herein in accordance with the common usage in the art, and is a nucleic acid sequence or sequences that act to direct transcription of an operably linked coding region. The promoter may also include or be operably linked to an enhancer element. An “enhancer” is a nucleotide sequence capable of increasing the transcriptional activity of an operably linked promoter.

A first nucleic acid sequence is operably linked with a second nucleic acid sequence when the sequences are placed in a functional relationship. For example, a coding sequence is operably linked to a promoter if the promoter activates the transcription of the coding sequence. Similarly, a promoter and an enhancer are operably linked when the enhancer increases the transcription of operably linked sequences. Enhancers may function when separated from promoters and as such, an enhancer may be operably linked to a promoter but may not be contiguous. Generally, however, operably linked sequences are contiguous.

In different embodiments, the enhancer may be a heterologous enhancer, meaning a nucleotide sequence which is not naturally operably linked to the particular promoter and which, when so operably linked, increases the transcriptional activity of the promoter. Reference to increasing the transcriptional activity is meant to refer to any detectable increase in the level of transcription of operably linked sequences compared to the level of the transcription observed with the particular promoter alone, as may be detected in standard transcriptional assays.

Thus, the promoter may be a cellular promoter that is typically found and expressed in the embryonic stem cell, including a promoter that is specific to embryonic stem cells, such as the oct-4 gene promoter, or a promoter for a constitutively active house-keeping gene such as the EF1-α gene promoter. The promoter may be a promoter that is specific to a particular differentiated cell type, or is a developmental specific promoter, meaning that the promoter is specific to a particular developmental stage in the organism from which the embryonic stem cell is derived, such as the nestin gene promoter. Alternatively, the promoter may be a viral promoter, for example, the immediate early promoter/enhancer from human cytomegalovirus, or may be a fusion promoter construct of a mammalian promoter and viral promoter and/or enhancer elements such as a CMV enhancer/human PDGF-beta promoter for neuron-specific transgene expression. Depending on the desired level of expression of the transgene, the promoter may be a basal level promoter, or it may be a strong promoter. The promoter may be constitutively active, or it may be an inducible promoter in response to particular environmental conditions or signals.

Baculoviral vectors tend to stay episomally in the nucleus and thus mediate transient gene expression. However, using a baculoviral vector containing an expression cassette encoding a dominant-selectable marker, stably transduced embryonic stem cells can be selected in a dose-dependent manner with respect to virus inoculum. With such techniques, the formation of stable transductants can be highly efficient, with frequency of colony formation up to 1 clone in 39 transduced cells (Condreay et al., 1999).

Therefore, the baculoviral vector may be designed for transient or stable expression of coding sequences contained in the nucleic acid molecule, for example a transgene, in the embryonic stem cell, for as long as the baculoviral vector is maintained in the cell and the promoter is active in the cell. Where such a transgene encodes a selectable marker, maintenance of selective pressure will assist in maintenance of the baculoviral vector and expression of the transgene. Such vectors may be suitable for in vitro applications, including separation of embryonic stem cells that have been differentiated or partially differentiated from undifferentiated embryonic stem cells in culture, and for cell culture research on differentiation of embryonic stem cells and developmental biological research.

However, for certain applications in which the embryonic stem cell is to be differentiated, or for ex vivo applications followed by in vivo implantation, stable integration of the nucleic acid molecule, including a nucleic acid molecule containing a transgene, into the chromosomes of the embryonic stem cell may be desired. Thus, the baculoviral vector may include sequences that direct integration of the nucleic acid molecule into the chromosomal DNA of the embryonic stem cell, preferably in a site-specific manner. For example, the baculoviral vector may include viral inverted terminal repeat sequences. Palombo and colleagues have developed a hybrid baculovirus-adeno associated virus (AAV) vector for site-specific integration in human cells (Palombo et al., 1998). This hybrid vector contains AAV inverted terminal repeats (ITRs) and the AAV rep gene for targeting and inserting a gene expression cassette into a defined region of human genome located on chromosome 19q13.3. In the present methods, in one example, a baculoviral vector designed to include the AAV inverted terminal repeats flanking the nucleic acid molecule and the AAV rep gene can be used for long-term expression of a transgene contained in the nucleic acid molecule in hES cells, due to site-specific chromosome integration mediated by the Rep/ITR system. The large cloning capacity of baculovirus allows for incorporation of the AAV Rep/ITR system into a single baculoviral vector in order to achieve targeted integration of exogenous DNA into the genome of an embryonic stem cell. The 4 Kb AAVS1 in human chromosome 19 constitutes at least part of a transcription unit (Kotin, et al. 1992).

Thus, in the present methods, the nucleic acid molecule is delivered to the embryonic stem cell by infection with a baculoviral vector described above. As used herein, the terms infection or infected refer to delivery by the virus of the genetic material to the cell, and are used interchangeably with the terms transduction or transduced in the present context, since the baculovirus is unable to replicate or produce viral proteins or infectious particles in mammalian cells.

The infection of the embryonic stem cell with the baculoviral vector may be performed using standard baculoviral techniques. For example, the infection may be performed in vitro by contacting the cell in culture with a viral preparation of the baculoviral vector, at an appropriate multiplicity of infection (MOI). For example, the viral concentration may be from an MOI of about 0.1 to an MOI of about 500, from an MOI of about 1 to an MOI of about 10 or of about 100 or of about 500.

To improve the gene transduction efficiency of baculovirus in embryonic stem cells, feeder cells may be excluded during viral infection. That is, if the embryonic stem cells are grown on a feeder cell support layer, as described in the examples below, the embryonic stem cell clumps may be removed from the feeder layer and transduced in suspension.

The above method of transducing an embryonic stem cell may be used to create an in vitro population of undifferentiated, partially or completely differentiated cells, useful for various in vitro purposes, including studying the differentiation pathways and mechanisms of certain cell types and the effects of certain compounds, cell factors or proteins on the differentiation or proliferation of embryonic stem cells, studying the ability of embryonic stem cells to produce and/or secrete growth factors, hormones or extracellular molecules that may themselves regulate the proliferation or differentiation of embryonic stem cells or other partially differentiated cell types, and also including drug screening methods in certain populations of undifferentiated, differentiated or partially differentiated cell types.

Thus, the above method can further include inducing the embryonic stem cell to differentiate after proliferation to produce progeny cells. Inducing differentiation is accomplished by exposing the embryonic stem cell to an additional cellular factor, such as particular growth factors, by withdrawal of serum or growth factors, such as FGF, insulin, transferrin, fibronectin, and/or by the addition of various nutrients or pharmacological reagents, such as selenium, dexamethasone, enzyme inhibitors and other compounds which can cause the cell to differentiate.

Once a desired level of proliferation is achieved, a population of cells expressing the transgene can be used to create a population of progeny cells for in vitro use or for in vivo therapeutic use. The amount of proliferation can be measured by cell counts and by talking a subset of cells and assaying for the incorporation of the thymidine analogue, BrdU.

A progeny cell is a cell derived from an embryonic stem cell which has undergone differentiation or partial differentiation, and which is more differentiated than the original embryonic stem cell. For example, an embryonic stem cell may differentiate to produce neuronal precursor cells, or neurons, or to produce pancreatic progenitor cells or pancreatic islet cells.

Embryonic stem cells that have been transduced or infected with a baculoviral vector containing a nucleic acid molecule of interest in accordance with the above methods, for example a therapeutic transgene, are useful for administering to a subject for treatment of a disease or disorder. In particular, the production of a population of embryonic stem cells that include a recombinant baculoviral nucleic acid is useful for embryonic stem cell therapy in which embryonic stem cells, or progeny cells derived from embryonic stem cells, are provided to a subject to replace or compensate for cells which have died or which do not function properly, thereby treating a disease or disorder.

Thus, the infection of the embryonic stem cell with the baculoviral vector may be carried out ex vivo for the purpose of transplantation into a subject. That is, the cells are transduced as described above, for example with a baculoviral vector carrying a therapeutic transgene, and then implanted in a subject. The transduced embryonic stem cells thus including a recombinant baculoviral nucleic acid may be implanted using surgical methods or by injection into a specific site. Alternatively, the stem cell may be allowed to divide and fully or partially differentiate in vitro into progeny cells, and such progeny cells may be implanted into a subject.

The embryonic stem cells can be tested to determine if they underwent differentiation by testing for the presence or absence of embryonic stem cell or differentiated cell markers. For example, neural precursor cells express the Nestin and Frizzled markers and should not express melanin, C-kit, GFAP, smooth muscle actin or neurofilament 160. In another example, cardiac precursor cells are Lin⁻ and C-kit⁺.

Thus, there is also presently provided a method of treating a disorder characterized by the premature death or malfunction of a specific cell type comprising administering an embryonic stem cell or a progeny cell that is a precursor cell for the specific cell type and which is proliferated by the above-described method, or a progeny cell that has been differentiated to become the specific cell type, to a subject. The cell is an embryonic stem cell that has been transduced with a baculoviral vector as described above, or is a progeny cell of such an embryonic stem cell, such that the cell has become genetically modified to express one or more therapeutic transgenes or therapeutic proteins or peptides.

A “disorder characterized by the premature death or malfunction of a specific cell type” refers to a disease or disorder in which a specific cell type in an individual has prematurely died, or which no longer, or never did, function at sufficient level so as to prevent the development of the disease or disorder, including insufficient levels of expression of a particular gene or gene product required to prevent development of the disease or disorder. In a healthy individual, such a specific cell type would be alive, including being in a quiescent or senescent state, and would function at a level which does not cause disease or a disorder, including expressing a particular gene or protein required to avoid or prevent development of disease or disorder. The disorder includes, but is not limited to, cancer including leukemia, neurological diseases such as Parkinson's Disease, Alzheimer's and ALS (Lou Gehrig's Disease), CNS damage including spinal cord injury and Multiple Sclerosis, cardiac damage, liver damage, kidney damage, pancreatic damage, retinal damage, intestinal damage, skeletal muscle damage including Muscular dystrophy, lung damage and diabetes.

Treating a disease or disorder refers to an approach for obtaining beneficial or desired results, including clinical results. Beneficial or desired clinical results can include, but are not limited to, alleviation or amelioration of one or more symptoms or conditions, diminishment of extent of disease, stabilization of the state of disease, prevention of development of disease, prevention of spread of disease, delay or slowing of disease progression, delay or slowing of disease onset, amelioration or palliation of the disease state, and remission (whether partial or total). “Treating” can also mean prolonging survival of a subject beyond that expected in the absence of treatment. “Treating” can also mean inhibiting the progression of disease, slowing the progression of disease temporarily, although more preferably, it involves halting the progression of the disease permanently.

The subject is any subject suffering from a disorder characterized by the premature death or malfunction of a specific cell type and who is in need of such treatment. The subject may be any animal, including a mammal, particularly a human.

The embryonic stem cell or progeny cells are administered to the subject by delivery to the site of the specific cell type which has prematurely died or which has malfunctioned, using methods known in the art, including by surgical implantation or by injection, for example at the site of a tissue or organ, such as the liver, pancreas, cardiac tissue, brain or spinal cord. For example, if the disorder being treated is diabetes, the embryonic stem cell or progeny cell may be a cell that can differentiate to become P islet cells, and may be implanted or injected in an islet in the pancreas. In another example, if the disorder being treated is cardiac damage, an embryonic stem cell or a progeny cell that can differentiate to become a cardiomyocyte may be implanted or injected into heart muscle. Similarly, in other examples embryonic stem cells can be differentiated into cells specific for lung, kidney, liver, intestinal wall, retinal or skeletal muscle tissue.

An effective amount of embryonic stem cells or progeny cells are administered to the subject. The term “effective amount” as used herein means an amount effective, at dosages and for periods of time necessary to achieve the desired result, for example, to treat the specific disorder.

The number of total embryonic stem or progeny cells to be administered will vary, depending on the disorder or disease to be treated, the type of cell that is administered, the mode of administration, and the age and health of the subject.

There is therefore also presently provided an embryonic stem cell, which comprises a recombinant baculoviral nucleic acid. The recombinant baculoviral nucleic acid may further comprise one or more therapeutic transgenes or a nucleic acid molecule encoding one or more therapeutic proteins or peptides.

To aid in administration, an embryonic stem cell, or a progeny cell differentiated from the embryonic stem cell, which comprises a recombinant baculoviral nucleic acid, may be formulated as an ingredient in a pharmaceutical composition. Therefore, in a further embodiment, there is provided a pharmaceutical composition comprising an embryonic stem cell, or a progeny cell differentiated from the embryonic stem cell, which comprises a recombinant baculoviral nucleic acid, and optionally a pharmaceutically acceptable diluent. The invention in one aspect therefore also includes such pharmaceutical compositions for use in treating a disorder characterized by the premature death or malfunction of a specific cell type. The compositions may routinely contain pharmaceutically acceptable concentrations of salt, buffering agents, preservatives and various compatible carriers. For all forms of delivery, the embryonic stem cell or progeny cell may be formulated in a physiological salt solution.

The pharmaceutical compositions may additionally contain other therapeutic agents useful for treating the particular disorder. Alternatively, the pharmaceutical composition may contain growth factors or cellular factors that facilitate cell survival and induce proliferation or differentiation of the embryonic stem cell or the progeny cell when delivered to the site of the disorder.

The proportion and identity of the pharmaceutically acceptable diluent is determined by chosen route of administration, compatibility with live cells, and standard pharmaceutical practice. Generally, the pharmaceutical composition will be formulated with components that will not kill or significantly impair the biological properties of the live embryonic stem cells or progeny cells.

The pharmaceutical composition can be prepared by known methods for the preparation of pharmaceutically acceptable compositions suitable for administration to subjects, such that an effective quantity of the embryonic stem cells or progeny cells, and any additional active substance or substances, is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the embryonic stem cells or progeny cells comprising a recombinant baculoviral nucleic acid, optionally in association with one or more pharmaceutically acceptable vehicles or diluents, and contained in buffer solutions with a suitable pH and iso-osmotic with physiological fluids.

The pharmaceutical composition may be administered to a subject in a variety of forms depending on the selected route of administration, as will be understood by those skilled in the art. The composition of the invention may be administered surgically or by injection to the desired site.

Solutions of the embryonic stem cells or the progeny cells may be prepared in a physiologically suitable buffer. Under ordinary conditions of storage and use, these preparations contain a preservative to prevent the growth of microorganisms, and that will maintain the live state of the cells. A person skilled in the art would know how to prepare suitable formulations. Conventional procedures and ingredients for the selection and preparation of suitable formulations are described, for example, in Remington's Pharmaceutical Sciences and in The United States Pharmacopeia The National Formulary (USP 24 NF19) published in 1999.

In different embodiments, the composition is administered by injection (subcutaneously, intravenously, intramuscularly, etc.) directly at the desired site where the cells that have prematurely died or are non-functional are located in the subject.

The dose of the pharmaceutical composition that is to be used depends on the particular condition being treated, the severity of the condition, the individual subject parameters including age, physical condition, size and weight, the duration of the treatment, the nature of concurrent therapy (if any), the specific route of administration and other similar factors that are within the knowledge and expertise of the health practitioner. These factors are known to those of skill in the art and can be addressed with minimal routine experimentation.

Also presently contemplated are uses of an embryonic stem cell for treating a disorder in a subject. Particularly contemplated is use of an embryonic stem cell comprising a recombinant baculoviral nucleic acid, for treating a disorder in a subject, the disorder characterized by the premature death or malfunction of a specific cell type in the subject. Such use includes use of the embryonic stem cell in the manufacture of a medicament for treating a disorder in a subject, the disorder characterized by the premature death or malfunction of a specific cell type in the subject.

The invention is further exemplified by the following non-limiting examples.

### EXAMPLES

### Example 1

Here we describe high-level expression of a transgene in baculovirus-infected human and mouse embryonic stem cells. We used baculoviruses harboring the human cytomegalovirus immediately early gene enhancer/promoter to infect suspended stem cells in a serum-free medium and obtained high transduction efficiency of in human embryonic stem cells. The infected stem cells kept proliferating, displayed no obvious changes in morphology and were able to differentiate into neurons.

### Materials and Methods:

Baculoviral vector Preparation: To generate baculoviral vectors with a reporter gene, the enhanced green fluorescent protein (eGFP) cDNA under the control of the human cytomegalovirus (CMV) immediately early gene promoter and enhancer was inserted into the transfer plasmid pFastBac1 (Gibco BRL, Life Technologies, Gaithersburg, Md., USA). The promoter was inserted between Not1 and Xba1, and the eGFP cDNA was between Xho1 and HindIII downstream of the promoters.

To generate baculoviruses that are able to integrate into the chromosomes in hES cells, a recombinant pFastBac1 was first constructed by inserting between the sites of Avr II and Sal I a fragment of pAAV plasmid that contain an expression cassette containing a multiple cloning site (MCS), a reporter gene encoding eGFP, a SV40 polyA signal, and two ITR sequences at both ends (Wang et al., 2005). The CMV promoter was then inserted between Kpn I and Hind III in the above recombinant pFastBac1. A DNA fragment containing the full sequences of Rep gene was amplified from pSub201, which was digested with Apa I to remove the encoding sequence of Cap gene and ligated again. The Rep gene was then digested with Rsr II and inserted into the recombinant pFastBac1, outside the ITRs in the antisense orientation with respect to the pPolh promoter.

Recombinant baculoviruses with the above expression cassettes were produced and propagated in Sf9 insect cells according to the manual of BAC-TO-BAC™ Baculovirus Expression system (Gibco BRL). Budded viruses in the insect cell culture medium were filtered through a 0.2 μm pore size filter (Minipore, Bedford, Mass., USA) to remove any contamination, and concentrated by ultracentrifugation at 25,000 g for 60 min. Viral pellets were re-suspended in appropriate volumes of 0.1 M phosphate-buffered saline (PBS) and their infectious titers (plaque-forming units, pfu) were determined by plaque assay on Sf9 cells.

mES and hES Culturing: Mouse embryonic stem cells line ES-D3 and mouse fibroblast STO were from American Type Culture Collection (ATCC, Manassas, Va.). ES-D3 cells were routinely grown on mitomycin-C (Sigma, Saint Luise, Mo.)-treated STO fibroblasts in Dulbecco's modified Eagle medium (DMEM) (Invitrogen, Carlsbad, Calif., USA) supplemented with 20% fetal bovine serum (FBS) (Hyclone, Logan, Utah), 0.1 mM 2-mercaptoethanol (Invitrogen), 10 ng/ml human recombinant leukemia inhibitory factor (hLIF) (Chemicon, Temecula, Calif.) and 50 U/ml penicillin/50 μg/ml streptomycin (Invitrogen). For subculture, ES-D3 cells were collected by gentle trypsination and replated on STO feeder layers that were seeded 24 to 48 h before.

Human embryonic stem cell line HES-1 and its feeder cell K₄ mouse embryonic fibroblasts (mEFs) were obtained from ES Cell International, Singapore. The Passage 4 mEFs were cultured to Passage 6 using DMEM containing 200 mM L-glutamine (Invitrogen), 10% FBS and 50 U/ml penicillin/50 μg/ml streptomycin. Passage 6 mEFs were mitotically inactivated by using 10 μg/ml mitomycin-C and replated into 0.1% gelatin (Sigma)-precoated centre well organ culture dish (BD Biosciences). HES-1 cells were then grown mEF layer in DMEM supplemented with 20% FBS, 0.1 mM Non-Essential Amino Acids (NEAA) (Invitrogen), 200 mM L-glutamine, 1% insulin-transferrin-selenium (ITS) (Invitrogen), 0.1 mM 2-mercaptoethanol and 50 U/ml penicillin/50 μg/ml streptomycin. The resulting hES colonies were then subcultured every 7 days by mechanical slicing and replating into fresh feeder layer.

Transduction of ES cells: For mES cell infection, ES-D3 cells were harvested by gentle trypsination and seeded 1 day before infection onto 6-well plate grown with mitomycin-C-treated STO cells. Shortly before infection, the medium with serum-free OPTI-MEM (Invitrogen) was replaced and concentrated recombinant baculoviruses were then added at desired multiplicity of infection (MOI). After 1 h incubation, the cells were washed with PBS and further incubated in normal ES-D3 medium for 1-3 d before observation under an inverted fluorescence microscope.

Two protocols were used for transduction of hES cells with baculovirus. In the first protocol, hES cells were infected when grown on mEFs. Briefly, hES cells were grown from cell clumps and maintained on mEF feeders in organ culture plate. On day 7 after subculture, 250 μl DMEM was used to replace the serum-containing medium; 250 μl recombinant baculovirus in PBS was added at MOI 50. After 1 h incubation, rinsed the cells with PBS and change back to normal culture medium. eGFP expression was examined under an inverted fluorescence microscope after 24 h.

In the second protocol, hES cell clumps in suspension were infected in order to eliminate viral absorption effects of mEFs. HES-1 cells were maintained on mEF feeder. At the time of routine passage, clumps of undifferentiated cells from hES colonies were isolated by mechanical slicing. For viral transduction, eight hES clumps were suspended in 50 μl DMEM and 50 μl baculovirus in PBS was added at MOI 100. After incubated for 2 h, the clumps were replated onto fresh mEF feeder. In the case of using baculoviruses containing the rep gene for gene transfer, hES clumps were treated with 10 mg/ml of dispase (Invitrogen) before viral infection.

Figure Legends:

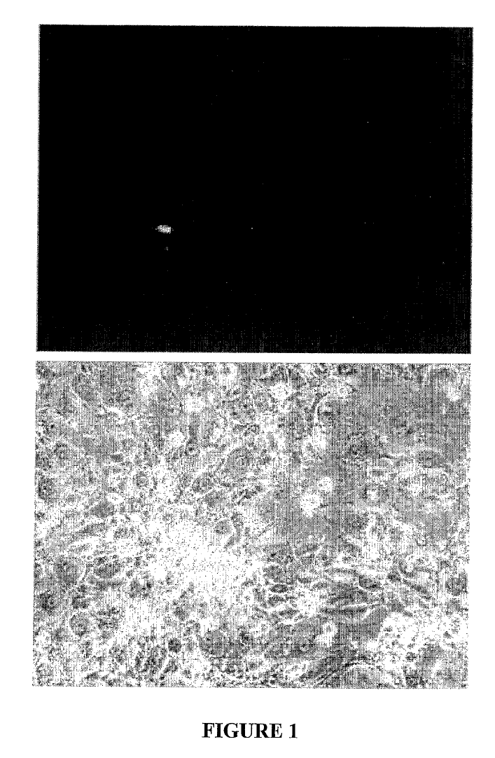

FIG. 1. Baculovirus-mediated transgene expression in ES-D3 cells grown on feeders. The mES cells were infected with recombinant baculovirus containing the eGFP gene under control of the CMV promoter at MOI 500. A fluorescence microscopy (top) and a phase-contrast (bottom) image of mES cells on day 3 after infection were shown.

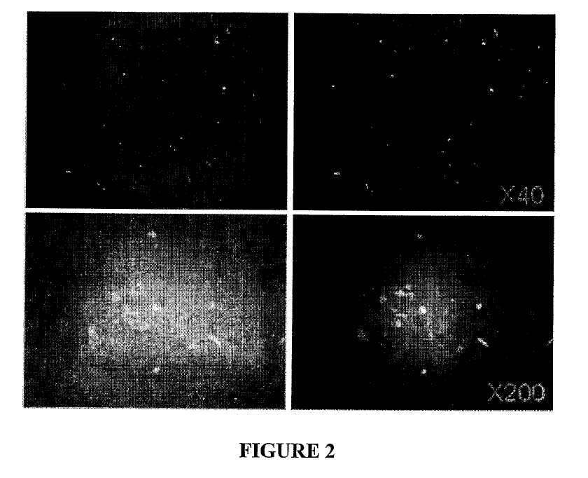

FIG. 2. Baculovirus-mediated transgene expression in HES-I cells grown on feeders. The hES cells grown on feeders were infected with recombinant baculovirus containing the eGFP gene under control of the CMV promoter at MOI 200. Overlapping of phase-contrast and fluorescence images (right) and fluorescence microscopy images (left) of a hES cell colony on day 1 after infection to show transduced cells. On the bottom, a high magnification of the hES cell colony to show transduced hES cells in the colony.

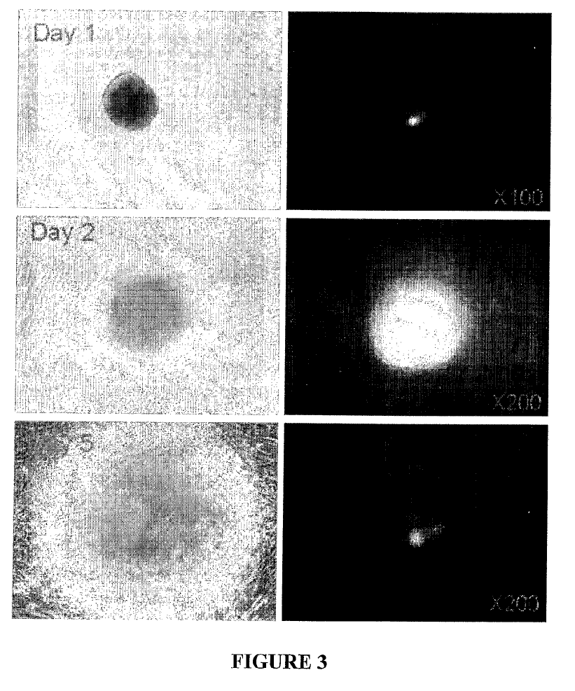

FIG. 3. Baculovirus-mediated transgene expression in HES-1 cell clumps. The hES cell clumps in suspension were infected with recombinant baculovirus containing the eGFP gene under control of the CMV promoter at MOI 100 and replated onto fresh mEF feeder. Phase-contrast images (left) and fluorescence images (right) of a hES cell colony from day 1 to 5 after infection were shown.

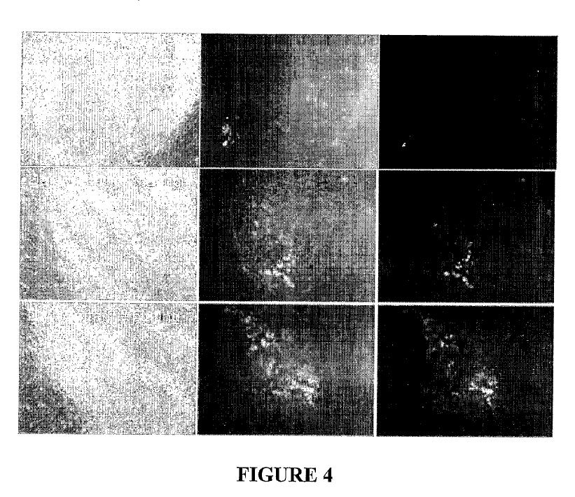

FIG. 4. Transgene expression in HES-1 cell clumps infected with hybrid baculoviruses containing the AAV rep genes and ITRs. The hES cell clumps in suspension were infected with the hybrid baculoviruses at an MOI 10 of pfu and re-plated onto fresh mEF feeder. Phase-contrast images (left), fluorescence images (right) and overlapping images (middle) of hES cell colonies were shown.

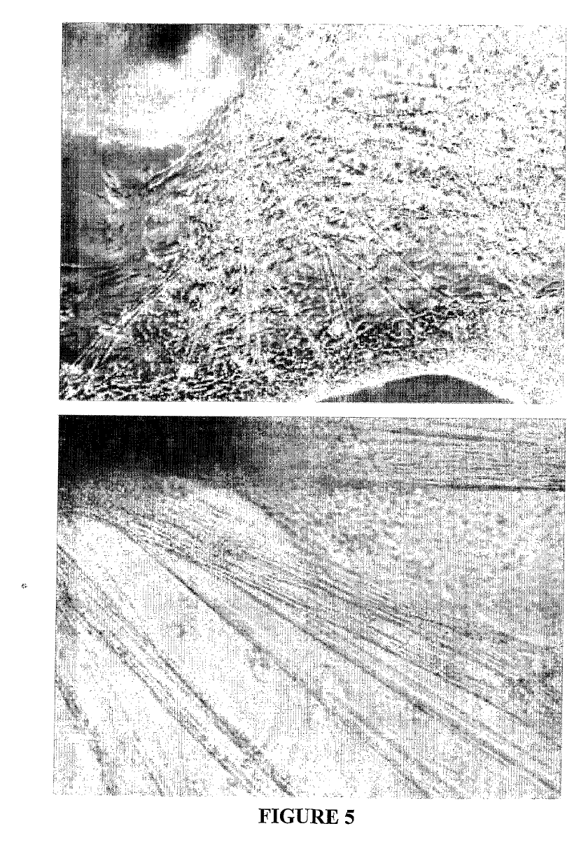

FIG. 5. Neuronal differentiation of baculovirus-infected hES cells. Ten days after viral infection at an MOI of 100 pfu, hES cells were induced to differentiate into neurons. Phase-contrast images of cell clumps with long neurites were shown.

Results:

ES-D3 cells were cultured on mouse embryonic fibroblasts and transduced with baculoviruses containing a eGFP reporter gene at MOI 200 and 500 in serum-free medium at 37° C. for 1 h. Expression of the transgene was observed 24 h after infection, with a efficiency of 1 to 2% in mouse ES cells after 1 d incubation. Intense transgene expression in the feeder layer due to absorption of viral particles was observed. No further increase in eGFP expression could be seen after incubation up to 3 d (FIG. 1).

The same batch of baculovirus preparation was also tested for gene transfer on human embryonic stem cell HES-1. At the beginning, a protocol similar to that used in mES transduction was used to infect hES cells grown on feeder layer. On day 1 after infection, mass transgene expression was observed in the feeder mEF cells at an MOI of 50 pfu (FIG. 2), suggesting the good quality of the virus preparation. However, almost no expression of eGFP was observed in the region of hES colonies at this time point. On day 2, there were a few cells with transgene expression in hES colonies, representing no more than 1% of the total hES cells (FIG. 2). Both infected mES and hES cells grown on feeder displayed a much lower percentage of transgene expression as comparing with that of feeder cells. This indicated the massive uptake of baculovirus by the mouse feeders.

To improve the gene transduction efficiency of baculoviruses in ES cells, feeder cells can be excluded during viral infection. By using a protocol that infected hES cell clumps in serum-free medium, a high percentage of infected hES cells with intensive eGFP expression was observed as early as day 1 after infection (FIG. 3). The transgene expression became more obvious on day 2 and, in several colonies, almost the whole colony was eGFP positive (FIG. 3). On day 3, new hES cells began to grow out from the center region of the colony, suggesting the baculovirus transduction process did not affect the proliferation of hES cells.

However, the eGFP signals were restricted only in the central part of the colony containing the original hES cell clump that was exposed to baculovirus infection. There was almost no eGFP expression in the peripheral part of the colony. This may be related to the transient feature of gene expression mediated by baculovirus or indicated that transgene could not be carried over to the newly generated hES cells. The number of eGFP-positive hES cells decreased over time, probably because the non-replicating transgene was diluted out with the proliferation of hES cells, although there were still several very bright transduced cells in the central regions of the HES colonies by day 5 (FIG. 3). The eGFP signals almost totally lost by day 7.

Stable transgene expression is required in many applications of genetically modified hES cells. We generated hybrid baculoviral vectors containing the rep 78/68 genes and inverted terminal repeats (ITRs) from adeno-associated virus (AAV) elements, the two AAV elements in responsible for viral DNA integration, and investigated whether the vectors could provide long-term transgene expression in hES cells. After the infection of hES cells with the hybrid viruses at a MOI of 10 pfu, no expression of eGPF was observed in the first 2 days. Weak eGFP expression started from day 3 onward and became obvious by day 6 (FIG. 4). The infected hES cell clumps were re-plated at day 7. After the re-plating, the number of eGFP-positive hES cells increased over time (FIG. 4). This differed strikingly from what observed in the experiments using baculoviruses without the AAV elements, indicating transgene duplication during hES cell proliferation. The eGFP-positive cells continued to express the transgene after 20 days of culture without any selection procedures.

Baculovirus infection caused no obvious cytotoxic effects on hES cells, even at a MOI of 200 pfu. Infected hES cells displayed no morphological changes and proliferated in a similar way as those non-infected control hES cells. Baculovirus infection did not affect expression of markers for embryonic stem cell pluripotency, as demonstrated by RT-PCR and Western blotting analyses (data not shown). When the infected hES cells were placed into a culture medium for neuronal differentiation, cells with long processes could be observed (FIG. 5), indicating that at least neuronal differentiation potency of hES cells was not affected by baculovirus infection.

### Example 2

Here we describe an efficient gene transfer method using baculoviral vectors for genetic modification of human embryonic stem (hES) cells, with transient transgene expression in up to 35% of hES cells. Using a hybrid baculoviral vector that employed adeno-associated virus (AAV) elements to mediate site-specific integration in human chromosome 19, we observed stable transgene expression during prolonged undifferentiated proliferation. The gene expression was well preserved in hES cells even after their differentiation.

Materials and Methods:

Baculoviral Vector Preparation: To generate baculoviral vectors carrying a repoter gene, the enhanced green fluorescent protein (eGFP) gene under control of human EF-1α promoter, the eGFP gene from vector pEGFP-C1 (Clontech) was PCR amplified and inserted between EcoRI and SpeI of pFastBac1 (Invitrogen). Then EF-1α promoter from pEF1V5-HisA (Invitrogen) was inserted between BamHI and EcoRI upstream of eGFP gene to the expression cassette.

To generate baculoviral vectors with the eGFP gene regulated by human cytomegalovirus (CMV) immediately early gene promoter and enhancer, the CMV promoter was inserted between Not1 and Xba1 and the eGFP gene was inserted between Xho1 and HindIII downstream of the promoter.

To generate baculoviral vectors that are able to facilitate the chromosome integration, a recombinant pFastBac1 was first constructed by inserting between Avr II and Sal I sites a fragment of pAAV plasmid that contains an expression cassette containing a multiple cloning site (MCS), a reporter gene encoding eGFP, a SV40 polyA signal, and two inverted terminal repeat (ITR) sequences at both ends (Wang, Guo et al. 2005). The CMV promoter was then inserted between Kpn I and Hind III in the above recombinant pFastBac1. A DNA fragment containing the full sequences of Rep gene was amplified from pSub201, which was digested with Apa I to remove the encoding sequence of Cap gene and ligated again. The Rep gene was then digested with Rsr II and inserted into the recombinant pFastBac1, outside the ITRs in an antisense orientation with respect to the pPolh promoter.

Recombinant baculoviral vectors with the above expression cassettes were produced and propagated in Sf9 insect cells according to the manual of BAC-TO-BAC™ Baculovirus Expression system (Invitrogen). Budded viruses in the insect cell culture medium were filtered through a 0.2 μm pore size filter (Millipore) to remove any contamination, and concentrated by ultracentrifugation at 25,000 g for 60 min. Viral pellets were re-suspended in appropriate volumes of 0.1 M phosphate-buffered saline (PBS) and their infectious titers (plaque-forming units, pfu) were determined by plaque assay on Sf9 cells.

hES Cell Culture and Transduction: Human embryonic stem cell line HES-1 (Reubinoff, Pera et al. 2000) and its feeder cell K₄ mouse embryonic fibroblasts (mEFs) were obtained from ES Cell International, Singapore. The Passage 4 mEFs were cultured to Passage 6 using DMEM (Invitrogen) supplemented with 2 mM L-glutamine (Invitrogen), 10% fetal bovine serum (FBS) (Hyclone), 50 U/ml penicillin, 50 μg/ml streptomycin. Passage 6 mEFs were mitotically inactivated by using 10 μg/ml mitomycin-C (Sigma) and replated into 0.1% gelatin (Sigma)-precoated center-well organ culture dishes (BD Biosciences). HES-1 cells were then grown mEF layer in DMEM supplemented with 20% FBS, 0.1 mM Non-Essential Amino Acids (NEAA) (Invitrogen), 2 mM L-glutamine, 1% insulin-transferrin-selenium (ITS) (Invitrogen), 0.1 mM 2-mercaptoethanol (Invitrogen), 50 U/ml penicillin and 50 μg/ml streptomycin. The hES colonies were then subcultured every 7 days by mechanical slicing and replating into fresh feeder layers.

hES cell clumps in suspension were used during transduction in order to eliminate the effects of mEFs. At the time of routine passage, hES cell clumps were isolated from hES colonies by mechanical slicing. For viral transduction, eight hES clumps were suspended in 50 μl DMEM and baculoviral vectors in 50 μL PBS were added at MOI of 100. After incubated for 2 h, the clumps were replated onto fresh mEF feeder.

FACS Analysis: To quantify the transduction efficiency of baculoviral vector on hES cells, various MOI were used. One day after transduction, hES cell clumps were detached from the mEFs by dispase digestion. The clumps were washed in PBS and trypsinized to make single cells. After washing with PBS, the cells were analyzed with FACSCalibur flow cytometer (Becton Dickinson) for the percentage of eGFP+ cells.

Derivation of Neurons from hES Cells: The derivation of neurons from hES cells was achieved based on the method described by Reubinoff et al (Reubinoff, et al. 2001). In brief, the hES cell differentiation was induced by prolonged culture for 3-4 weeks on feeders. The distinct areas with uniformly white-gray and opaque appearance under dark-field stereomicroscope were cut and dissected into small cell clumps. These cell clumps were then transferred to low cell binding six-well plate (NUNC) containing DMEM/F12 (1:1) (Invitrogen) supplemented with B27 (1:50) (Invitrogen), 2 mM L-glutamine, 50 U/ml penicillin, 50 μg/ml streptomycin, 20 ng/ml hEGF (Chemicon) and 20 ng/ml bFGF (Chemicon). After culture of 1-3 weeks, round neural spheres were formed. The spheres were then plated into poly-D-lysine (Sigma) and laminin (Sigma) coated dish. Neuronal differentiation was induced by the withdrawal of grow factors from the culture medium.

hES Cell Differentiation In Vitro: To form embryoid bodies (EBs), hES cells were grown to form large colonies and detached by using 0.1 mg/ml dispase. The hES cell clumps were transferred to a 15 ml conical tube containing 10 ml differentiation medium [80% knock-out DMEM (Invitrogen), 20% FBS, 2 mM L-glutamine and 0.1 mM NEAA] and allowed to settle to the bottom. The supernatant was removed. The cell clumps were resuspended in differentiation medium and transferred to a Petri dish. The cells were fed every day by replacing half of the medium with fresh differentiation medium and cultured for 1 week (Itskovitz-Eldor, et al. 2000).

RT-PCR Analysis: Total RNA was extracted from hES cells or EBs using an RNAeasy™ kit (Qiagen) according to the manufacturer's instructions. First-strand cDNA was synthesized using SuperScript III First-Strand Synthesis System™ for RT-PCR (Invitrogen). 1 μliter of cDNA reaction mix was subjected to PCR amplification with primers shown in the following Table 1. Reactions were subjected to 30 PCR cycles after denaturation at 94° C. for 4 min as following: 94° C. for 30 s; 55° C. for 30 s; 72° C. for 60 s. An extension step of 72° C. for 5 min was included. All products were electrophoresised on a 2% agarose gel.

TABLE 1
RT-PCR Primers

| | | | |
|---|---|---|---|
| | Forward Primer/ | Product | SEQ |
| Gene | Reverse Primer | Size | ID NO: |

| | | | |
|---|---|---|---|
| Oct-4 | (Forward Primer) | 169 bp | 1 |
| | CTTGCTGCAGAAGTGGGTGGAGGAA | | |
| | (Reverse Primer) | | 9 |
| | CTGCAGTGTGGGTTTCGGGCA | | |
| Nanog | (Forward Primer) | 426 bp | 2 |
| | GCGCGGTCTTGGCTCACTGC | | |
| | (Reverse Primer) | | 10 |
| | GCCTCCCAATCCCAAACAATACGA | | |
| β-actin | (Forward Primer) | 200 bp | 3 |
| | CGCACCACTGGCATTGTCAT | | |
| | (Reverse Primer) | | 11 |
| | TTCTCCTTGATGTCACGCAC | | |
| PAX6 | (Forward Primer) | 309 bp | 4 |
| | CGTCCATCTTTGTTGGGAAATC | | |
| | (Reverse Primer) | | 12 |
| | GAGCCTCATCTGAATCTTCTCCG | | |
| NeuroD | (Forward Primer) | 576 bp | 5 |
| | GAGACTATCACTGCTCAGGA | | |
| | (Reverse Primer) | | 13 |
| | GATAAGCCCTTGCAAAGCGT | | |
| T | (Forward Primer) | 284 bp | 6 |
| | CAACCACCGCTGGAAGTAC | | |
| | (Reverse Primer) | | 14 |
| | CCGCTATGAACTGGGTCTC | | |
| Globin | (Forward Primer) | 397 bp | 7 |
| | GACTGAGAGGACCATCATTG | | |
| | (Reverse Primer) | | 15 |
| | TCAGGACAGAGGATACGACC | | |
| AFP | (Forward Primer) | 675 bp | 8 |
| | AGAACCTGTCACAAGCTGTG | | |
| | (Reverse Primer) | | 16 |
| | GACAGCAAGCTGAGGATGTC | | |

Immunocytochemistry Studies: lES cell colonies were washed with PBS and fixed at room temperature with 4% paraformaldehyde for 10 min and permeated with 1% saponin for 10 min. The cells were then blocked with 5% normal goat serum for 30 min and then incubated with mouse anti-Oct-4 monoclonal antibody (Chemicon) or mouse anti-SSEA-4 monoclonal antibody (Santa Cruz Biotechnology) for overnight at 4° C. Goat anti-mouse IgG-cy3 antibody (Sigma) was then applied for 60 min to visualize the antigens.

Figure Legends:

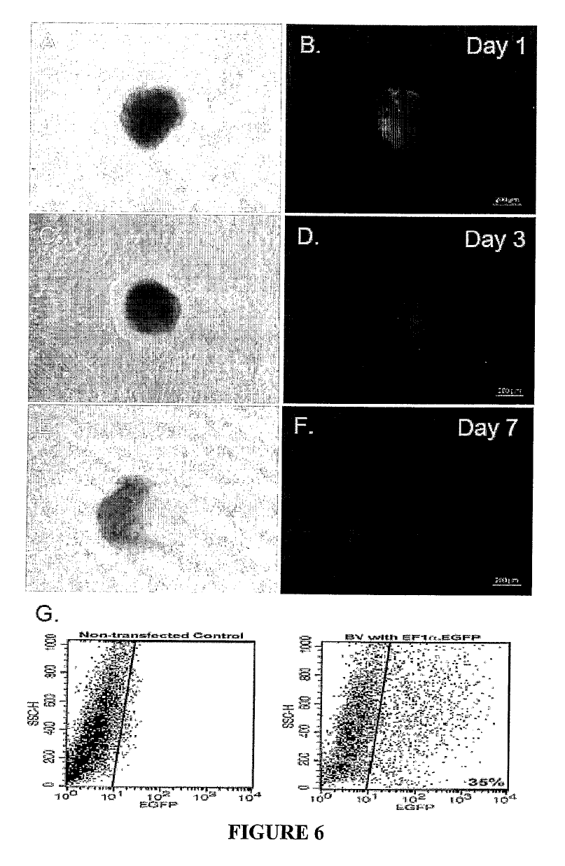

FIG. 6. Transient transgene expression in hES cells mediated by baculoviral vectors. (A-F) A time-course observation shows a hES cell colony 1 (A, B), 3 (C, D) and 7 (E, F) days after transduction with recombinant baculovirus carrying eGFP gene under the control of the EF1α promoter. Phase-contrast (A, C, E) and fluorescence images (B, D, F) are shown. (G) Flow cytometric analysis of the percentage of eGFP positive hES cells at 2 days post-transduction.

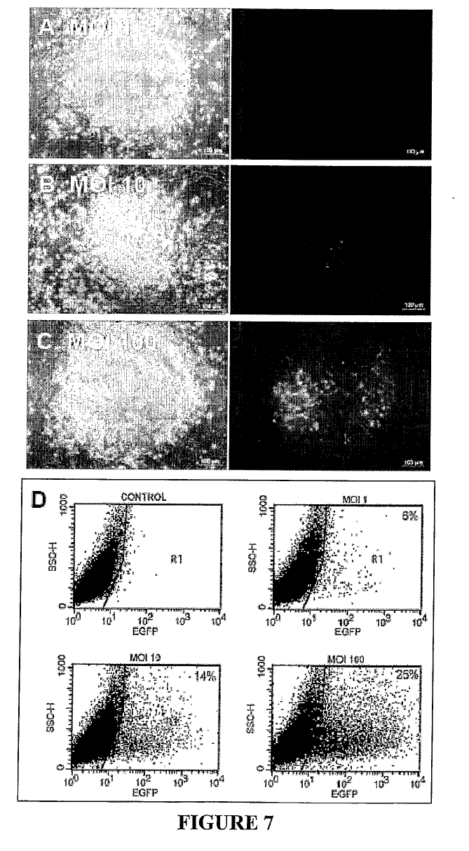

FIG. 7. Dose-dependant transduction efficiency by baculoviral vectors with the CMV promoter in hES cells. (A-C) The hES cell clumps isolated by mechanical slicing were transduced with recombinant baculoviruses at various MOIs from 1 to 100 pfu in suspension after dispase digestion. Phase-contrast images (left) and fluorescence images (right) of hES cell colonies 1 day after transduction were shown. (D) Dose-dependant transduction efficiency by baculoviral vectors in hES cells. FASC analysis showed the percentages of transgene expression in hES cells 1 day after transduction with recombinant baculoviruses at various MOI.

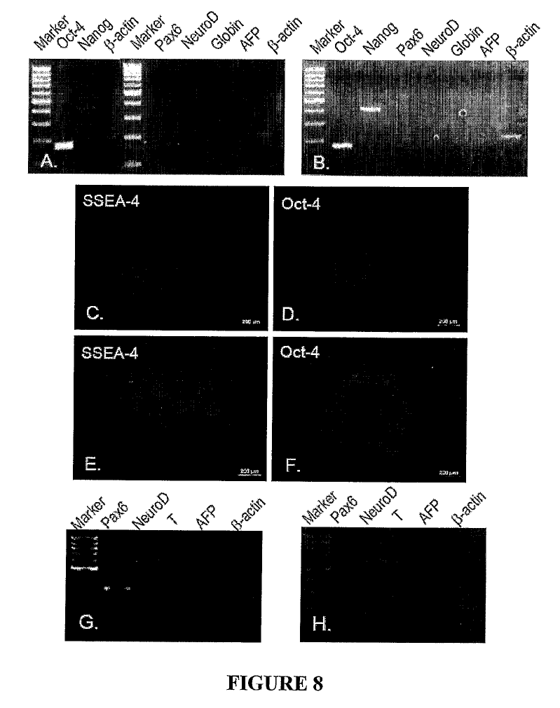

FIG. 8. Expression of molecular markers in hES cells after baculoviral vector transduction. The hES cell clumps were transduced with recombinant baculovirus containing the eGFP gene under control of the CMV promoter at MOI of 100 and replated onto fresh mEF feeders. Seven days after transduction, RT-PCR was used to detect the expression of molecular markers in mock-transduced hES cells (A) and hES cells transduced by baculoviral vectors (B), and immunostaining to detect the expression of SSEA-4 and Oct-4 markers in mock-transduced hES cells (C, D) and hES cells transduced by baculoviral vectors (E, F). The transduced hES cells were also used to generate embryoid bodies. Seven days after embryoid body formation, RT-PCR showed the expression of markers for three germ layers in embryoid bodies derived from mock-transduced hES cells (G) and from hES cells transduced by baculoviral vectors (H).

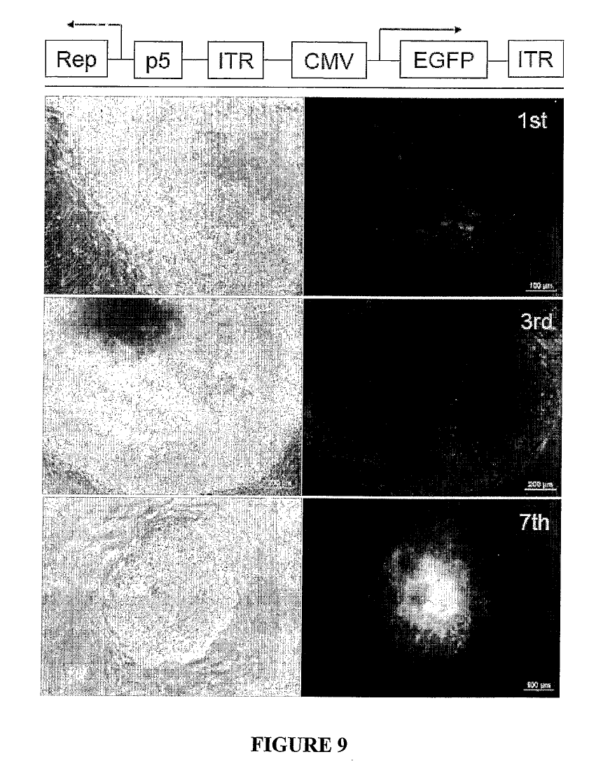

FIG. 9. Stable transgene expression in hES cells mediated by hybrid bacuolviral vectors. The hES cell clumps were transduced with hybrid baculoviruses containing the AAV rep gene and ITRs and replated onto fresh feeders. EGFP positive hES cell were isolated mechanically and replated onto fresh feeders every week at the time of normal subculture. The Rep/ITR construct that directs transgene integration is shown on the top. Phase-contrast (left) and fluorescence (right) images demonstrate the enrichment of eGFP positive hES cells after 1st, 3rd and 7th selection.

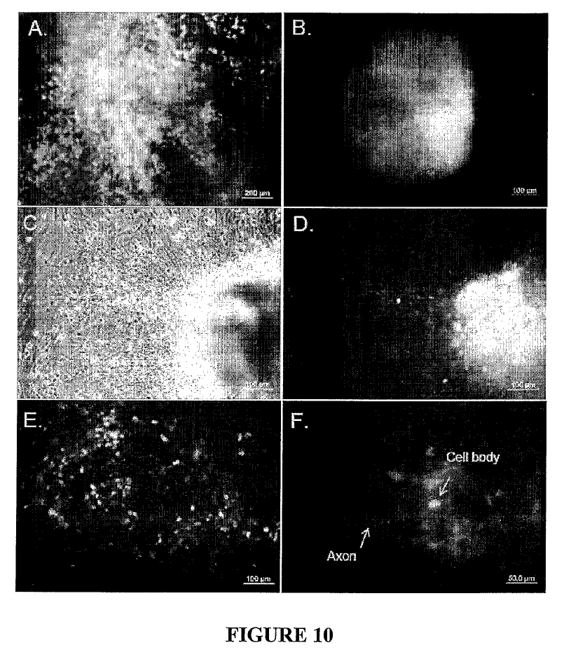

FIG. 10. Neural differentiation of hES cells stably transduced by hybrid baculoviral vectors. The stably transduced hES cells were directed to differentiate into neurons. (A) A fluorescence image of the overgrowth of eGFP positive hES cells on feeders for 4 weeks without subculture. (B) A fluorescence image of neural sphere generated from the overgrown hES cells. (C) A phase-contrast image and (D) a fluorescence image show typical neural differentiation from neural sphere derived from the stable hES cells after plating for 2 weeks. (E & F) Fluorescence images of typical neurons derived from the stably transduced hES cells with eGFP in both their cell bodies and neurites.

Results:

We isolated hES cell clumps of HES-1 line by mechanical slicing, suspended them in serum-free hES cell culture medium and transduced the clumps with baculoviral vectors containing an enhanced green fluorescent protein (eGFP) gene at a multiplicity of infection (MOI) of 100 plague-forming units (pfa) for 1 h before replating on feeders. Driven by the human EF1α promoter, eGFP expression was observed as early as 6 h after transduction and became highly intensive at 24 h, with bright green fluorescence covering the whole clumps (FIGS. 6A & B). The transduced hES cells kept outgrowing from the clumps, resulting a colony with a central ‘green’ region surrounded by newly generated, eGFP-negative hES cells (FIGS. 6C & D). The eGFP signals in the central region decreased over time and became very weak by day 7 (FIGS. 6E & F). Flow cytometric analysis demonstrated about 35% of the hES cells in the clumps being eGFP positive at day 2 (FIG. 6G). Similar results were observed when baculoviral vectors with the human cytomegalovirus immediately early gene promoter and enhancer (CMV promoter) were used (FIG. 7). These findings suggest that baculoviral vectors are competent in mediating transient gene expression in hES cells.

Baculovirus transduction caused no obvious cytotoxic effects on hES cells, even at an MOI of 1000 pfu. Infected hES cells displayed no morphological changes and proliferated in a similar way as those non-infected control hES cells. Baculovirus infection had no effects on expression of markers for embryonic stem cell pluripotency such as Oct-4, SSEA-4 and nanog and did not induce the expression of the representative markers for the three germ layers, including Pax6, NeuroD, globin and AFP (FIG. 8). This observation indicates that baculoviral infection does not affect the pluripotency and proliferation of hES cells.

Stable transgene expression in hES cells after genetic manipulation is crucial for the applications of these hES cells in regenerative medicine that require long-term expression of transgenes. To extend the duration of baculoviral vector-mediated transgene expression, we constructed a hybrid baculoviral vector by including the rep 78/68 genes and inverted terminal repeat (ITR) sequences from adeno-associated virus (AAV) (Palombo, Monciotti et al. 1998). This chimeric virus construct takes advantage of Rep-mediated site-specific integration at the AAVS1 site in human chromosome 19 (19q13.3-qter) by non-homologous recombination (McCarty, et al. 2004). The constitutively active CMV promoter was used in order to observe eGFP expression in both undifferentiated and differentiated cells. Transduced with this hybrid baculoviral vector, hES cells displayed almost no eGFP expression in the first 2 days. EGFP-positive hES cells begun to appear in small number from day 3 and became obvious at day 7, by when these eGFP-positive cells were isolated by mechanical slicing and replated on fresh feeders. The number of eGFP-positive hES cells in the colonies increased with each round of mechanical selection and after 6 to 8 selections almost the whole hES colony became eGFP positive (FIG. 9). The selected eGFP positive hES cells continued to express the transgene for at least 10 passages without transgene silencing, suggesting chromosome integration of transgene mediated by the hybrid vector.

To further examine the stability of transgene expression, we investigated whether the eGFP expression could be preserved after differentiation into neurons, one type of the representative cells from ectoderm. First, the eGFP positive hES cells were overgrown on feeders for 4 weeks (FIG. 10A). Then, neural spheres were generated from the overgrown cells by incubating in suspension in the presence of bFGF and EGF for 1-3 weeks (Reubinoff, et al. 2001). The resulting neural spheres remained intense eGFP fluorecence (FIG. 10B). After plating on poly-D-lysine and laminin coated dish and cultured in the absence of growth factors, the neural sphere displayed typical neural differentiation phenotype with extending neurits and expressed bright eGFP with little silencing (FIG. 10C-F). The transgene expression was maintained in the neurons for at least 50 days (the end of the experiment). Besides the neurons, cardiomyocytes, a representative type of cells from mesoderm, were also derived from these transduced hES cells during spontaneous differentiation. Importantly, these cardiomyocytes remained eGFP positive after the differentiation.

As can be understood by one skilled in the art, many modifications to the exemplary embodiments described herein are possible. The invention, rather, is intended to encompass all such modification within its scope, as defined by the claims.

All documents referred to herein are fully incorporated by reference.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

### REFERENCES

Ho, Y. C., Y. C. Chung, et al. (2005). “Transgene expression and differentiation of baculovirus-transduced human mesenchymal stem cells.” J Gene Med 7(7): 860-8.
Kawabata, K., F. Sakurai, et al. (2005). “Efficient gene transfer into mouse embryonic stem cells with adenovirus vectors.” Mol Ther 12(3): 547-54.
Kost, T. A., J. P. Condreay, et al. (2005). “Baculovirus as versatile vectors for protein expression in insect and mammalian cells.” Nat Biotechnol 23(5): 567-75.
Kotin, R. M., R. M. Linden, et al. (1992). “Characterization of a preferred site on human chromosome 19q for integration of adeno-associated virus DNA by non-homologous recombination.” Embo J 11(13): 5071-8.
McCarty, D. M., S. M. Young, Jr., et al. (2004). “Integration of adeno-associated virus (AAV) and recombinant AAV vectors.” Annu Rev Genet 38: 819-45.
Palombo, F., A. Monciotti, et al. (1998). “Site-specific integration in mammalian cells mediated by a new hybrid baculovirus-adeno-associated virus vector.” J Virol 72(6): 5025-34.
Pfeifer, A., M. Ikawa, et al. (2002). “Transgenesis by lentiviral vectors: lack of gene silencing in mammalian embryonic stem cells and preimplantation embryos.” Proc Natl Acad Sci USA 99(4): 2140-5.
Reubinoff, B. E., P. Itsykson, et al. (2001). “Neural progenitors from human embryonic stem cells.” Nat Biotechnol 19(12): 1134-40.
Capecchi M R. Generating mice with targeted mutations. Nat Med. 2001; 7(10): 1086-90.
Cereseto A, Giacca M. Integration site selection by retroviruses. AIDS Rev. 2004 January-March; 6(1):13-21.
Condreay J P, Witherspoon S M, Clay W C, Kost T A. Transient and stable gene expression in mammalian cells transduced with a recombinant baculovirus vector. Proc Natl Acad Sci USA. 1999; 96(1): 127-32.
Downing G J, Battey J F Jr. Technical assessment of the first 20 years of research using mouse embryonic stem cell lines. Stem Cells. 2004; 22(7): 1168-80.
Eiges R, Schuldiner M, Drukker M, Yanula O, Itskovitz-Eldor J, Benvenisty N. Establishment of human embryonic stem cell-transfected clones carrying a marker for undifferentiated cells. Curr Biol. 2001; 11(7): 514-8.
Ghosh S et al. Baculovirus as mammalian cell expression vector for gene therapy: an emerging strategy. Mol Ther 2002; 6: 5-11.
Gropp M, Itsykson P, Singer O, Ben-Hur T, Reinhartz E, Galun E, Reubinoff B E. Stable genetic modification of human embryonic stem cells by lentiviral vectors. Mol Ther. 2003; 7(2): 281-7.
Hacein-Bey-Abina S, Von Kalle C, Schmidt M, McCormack M P, Wulffraat N, Leboulch P, Lim A, Osborne C S, Pawliuk R, Morillon E, Sorensen R, Forster A, Fraser P, Cohen J I, de Saint Basile G, Alexander I, Wintergerst U, Frebourg T, Aurias A, Stoppa-Lyoimet D, Romana S, Radford-Weiss I, Gross F, Valensi F, Delabesse E, Macintyre E, Sigaux F, Soulier J, Leiva L E, Wissler M, Prinz C, Rabbitts T H, Le Deist F, Fischer A, Cavazzana-Calvo M. LMO2-associated clonal T cell proliferation in two patients after gene therapy for SCID-X1. Science. 2003 Oct. 17; 302(5644):415-9.
Kost T A, Condreay J P. Recombinant baculoviruses as mammalian cell gene delivery vectors. Trends Biotechnol 2002; 20: 173-180.
Lakshmipathy U, Pelacho B, Sudo K, Linehan J L, Coucouvanis E, Kaufman D S, Verfaillie C M. Efficient transfection of embryonic and adult stem cells. Stem Cells. 2004; 22(4): 531-43.
Ma Y, Ramezani A, Lewis R, Hawley R G, Thomson J A. High-level sustained transgene expression in human embryonic stem cells using lentiviral vectors. Stem Cells. 2003; 21(1): 111-7.
O'Reilly, D. R., Miller, L. K. and Luchow, V. A. (1992) Baculovirus Expression Vectors: A Laboratory Manual (W.H. Freeman and Company, New York).
Smith-Arica J R, Thomson A J, Ansell R, Chiorini J, Davidson B, McWhir J. Infection efficiency of human and mouse embryonic stem cells using adenoviral and adeno-associated viral vectors. Cloning Stem Cells. 2003; 5(1):51-62.
Shoji I, Aizaki H, Tani H, Ishii K, Chiba T, Saito I, Miyamura T, Matsuura Y. Efficient gene transfer into various mammalian cells, including non-hepatic cells, by baculovirus vectors. J Gen Virol. 1997; 78 (Pt 10):2657-64.
Wang C Y, Guo H Y, Lim T M, Ng Y K, Neo H P, Hwang P Y, Yee W C, Wang S. Improved neuronal transgene expression from an AAV-2 vector with a hybrid CMV enhancer/PDGF-beta promoter. J Gene Med. 2005 Mar. 9; [Epub ahead of print]
Zwaka T P, Thomson J A. Homologous recombination in human embryonic stem cells. Nat Biotechnol. 2003; 21(3): 319-21.
Sarkis C, Serguera C, Petres S, Buchet D, Ridet J L, Edelman L, Mallet J. Efficient transduction of neural cells in vitro and in vivo by a baculovirus-derived vector. Proc Natl Acad Sci USA. 2000; 97(26):14638-43.
Itskovitz-Eldor J, Schuldiner M, Karsenti D, Eden A, Yanuka O, Amit M, Soreq H, Benvenisty N. Differentiation of human embryonic stem cells into embryoid bodies compromising the three embryonic germ layers. Mol Med. 2000; 6(2):88-95.

1. A method of delivering a nucleic acid molecule to an embryonic stem cell, comprising infecting the embryonic stem cell with a baculoviral vector, the baculoviral vector comprising the nucleic acid molecule.

2. The method of claim 1 wherein the embryonic stem cell is a human embryonic stem cell.

3. The method of claim 1 wherein the nucleic acid molecule comprises a transgene, the transgene including a coding region and an operably linked promoter.

4. The method of claim 3 wherein the transgene is a reporter gene, a selectable marker gene, a gene involved in differentiation of embryonic stem cells or a therapeutic transgene.

5. The method of claim 3 wherein the promoter is a promoter specific to embryonic stem cells, a promoter specific to a differentiated cell type, a developmental specific promoter, a viral promoter, or a fusion promoter of a mammalian promoter and a viral promoter and/or enhancer.

6. The method of claim 5 wherein the promoter is the human cytomegalovirus immediate early promoter/enhancer.

7. The method of claim 1 wherein the baculovirus vector further comprises inverted terminal repeat (ITR) sequences flanking the nucleic acid molecule, and a rep gene.

8. The method of claim 7 wherein the ITR sequences and the rep gene are the adeno-associated virus ITR sequences and rep gene.

9. The method of claim 1 in which the baculoviral vector is a Autographa californica multiple nucleopolyhedrovirus vector.

10. The method of claim 1 further comprising inducing the embryonic stem cell to differentiate.

11. A recombinant baculoviral nucleic acid comprising a promoter specific to embryonic stem cells.

12. The recombinant baculoviral nucleic acid of claim 11 wherein the promoter specific to embryonic stem cells is operably linked to a coding region or to a multiple cloning site.

13. The recombinant baculoviral nucleic acid of claim 11 wherein the promoter specific to embryonic stem cells is the oct-4 promoter.

14. An embryonic stem cell comprising a recombinant baculoviral nucleic acid of claim 11.

15. A pharmaceutical composition comprising an embryonic stem cell of claim 14.

16. A method of treating a disorder characterized by the premature death or malfunction of a specific cell type comprising administering to a subject an embryonic stem cell comprising a recombinant baculoviral nucleic acid.

17. The method of claim 16 further comprising inducing the embryonic stem cell to differentiate prior to administering.

18. The method of claim 16 wherein the baculoviral vector comprises a therapeutic transgene.

19. The method of claim 16 wherein the disorder is cancer, leukemia, Parkinson's disease, Alzheimer's disease, ALS, CNS damage, spinal cord injury, Multiple Sclerosis, cardiac damage, liver damage, kidney damage, pancreatic damage, retinal damage, intestinal damage, skeletal muscle damage, Muscular dystrophy, lung damage or diabetes.

20. The method of claim 16 wherein the administering comprises surgical implantation or injection.

21. The method of claim 16 further comprising treating the embryonic stem cell with an additional growth factor prior to administering.

22. The method of claim 21 wherein the additional growth factor is fibroblast growth factor or a neurotrophin.

23-30. (canceled)


SEQUENCE LISTING (16 sequences)

SEQ ID NO: 1 - DNA, 25 bp, artificial
  synthetic primer
cttgctgcagaagtgggtggaggaa

SEQ ID NO: 2 - DNA, 20 bp, artificial
  synthetic primer
gcgcggtcttggctcactgc

SEQ ID NO: 3 - DNA, 20 bp, artificial
  synthetic primer
cgcaccactggcattgtcat

SEQ ID NO: 4 - DNA, 22 bp, artificial
  synthetic primer
cgtccatctttgttgggaaatc

SEQ ID NO: 5 - DNA, 20 bp, artificial
  synthetic primer
gagactatcactgctcagga

SEQ ID NO: 6 - DNA, 19 bp, artificial
  synthetic primer
caaccaccgctggaagtac

SEQ ID NO: 7 - DNA, 20 bp, artificial
  synthetic primer
gactgagaggaccatcattg

SEQ ID NO: 8 - DNA, 20 bp, artificial
  synthetic primer
agaacctgtcacaagctgtg

SEQ ID NO: 9 - DNA, 21 bp, artificial
  synthetic primer
ctgcagtgtgggtttcgggca

SEQ ID NO: 10 - DNA, 24 bp, artificial
  synthetic primer
gcctcccaatcccaaacaatacga

SEQ ID NO: 11 - DNA, 20 bp, artificial
  synthetic primer
ttctccttgatgtcacgcac

SEQ ID NO: 12 - DNA, 23 bp, artificial
  synthetic primer
gagcctcatctgaatcttctccg

SEQ ID NO: 13 - DNA, 20 bp, artificial
  synthetic primer
gataagcccttgcaaagcgt

SEQ ID NO: 14 - DNA, 19 bp, artificial
  synthetic primer
ccgctatgaactgggtctc

SEQ ID NO: 15 - DNA, 20 bp, artificial
  synthetic primer
tcaggacagaggatacgacc

SEQ ID NO: 16 - DNA, 20 bp, artificial
  synthetic primer
gacagcaagctgaggatgtc